(19) United States
(12) Patent Application Publication
(10) Pub. No.: US 2008/0097332 A1
(43) Pub. Date: Apr. 24, 2008

(54) SURGICAL ACCESS PORT

(75) Inventors: Ellis Skott Greenhalgh, Wyndmoor, PA (US); Michael Paul Igoe, Perkasie, PA (US)

Correspondence Address: LEVINE BAGADE HAN LLP, 2483 EAST BAYSHORE ROAD, SUITE 100, PALO ALTO, CA, 94303, US

(73) Assignee: Stout Medical Group L.P., Perkasie, PA (US)

(21) Appl. No.: 11/874,855

(22) Filed: Oct. 18, 2007

Related U.S. Application Data
(60) Provisional application No. 60/852,498, filed on Oct. 18, 2006.

(51) Int. Cl. A61M 39/04

(52) U.S. Cl. 604/167.06

(57) ABSTRACT

This disclosure concerns self sealing access ports, and especially ports usable to provide access to body cavities for surgical procedures. A self-sealing surgical access port permitting a surgical tool to be used within a body cavity, for example during laparoscopic surgery, is disclosed. The tube can be elastically deformable radially outwardly to permit the surgical tool to pass through the duct and into the body cavity.

### CROSS-REFERENCE TO RELATED APPLICATIONS

This application seeks priority to U.S. Provisional Application No. 60/852,498 filed Oct. 18, 2006 which is incorporated by reference herein in its entirety.

### TECHNICAL FIELD

This disclosure concerns sell sealing access ports, and especially ports usable to provide access to body cavities for surgical procedures.

### BACKGROUND

Laparoscopy and laparoscopic surgical techniques allow various abdominal organs such as the liver, gallbladder, spleen, peritoneum, diaphragm, as well as portions of the colon and small bowel to be readily visualized and operated upon. For example, lesions on an organ may be biopsied, an organ may be sectioned, and contrast material may be injected into the organ to assist in the visualization of vascular as well as other systems.

During such procedures, the abdominal cavity is inflated with a gas such as air or nitrous oxide to create a working space in which laparoscopic surgical tools and cameras may be deployed to effect examination and various surgical procedures. Such tools may include, for example, scissors, scalpels, clamps, syringes and electro-coagulation devices to control bleeding.

It is clear from the above description that if surgical tools are to be inserted, manipulated and withdrawn from the outside of an abdominal cavity that is expanded using internal pressure, there must be a port which provides access to the cavity while also maintaining the inflation pressure within the cavity during insertion, manipulation and removal or the tools during the surgical procedures.

In addition to providing access to the abdominal cavity while maintaining a substantially fluid tight seal during the insertion, removal and manipulation of surgical tools, the access port should also have acceptable characteristics for snag resistance and push through and removal force. Snag resistance refers to the propensity of surgical tools to catch or snag on the surface of a seal, and not slide smoothly over it. If the seal surface is prone to snagging the tool, it can lead to seal damage, such as tears that compromise the fluid tightness of the seal. Push through aid removal forces refer to the manual force necessary to insert or remove a tool through the access port. Excessive insertion or removal force, which can be caused by snagging or by too tight a contact force between engaging surfaces effecting the fluid tight seal, is to a be avoided as it also may lead to seal damage, patient injury, as well as increase the overall difficulty of performing the procedure. There is clearly a need for a surgical access port that provides a substantially fluid tight seal while maintaining adequate snag resistance characteristics and acceptable push through and removal force requirements.

### SUMMARY

A self-sealing surgical access port permitting a surgical tool to be used within a body cavity, for example during laparoscopic surgery, is disclosed. The access port can comprise a rigid duct or cannula that can have a distal end positionable within the body cavity. The duct can be extendable through living tissue of the body and can have a proximal end positionable outside of the cavity. A flexible tube can be positioned substantially coaxially within the duct and be attached thereto. The tube can have an inner low-friction surface surrounded by and elastic membrane. The membrane can be biased so as to form a constructed region of the tube. The tube can be elastically deformable radially outwardly to permit the surgical tool to pass through the duct and into the body cavity. The constricted region of the membrane closes around the tool to substantially continuously seal the tube while the tool extends therethrough. The tube can be made to not form a seal in the absence of a tool extending through lithe duct, in which case the duct can be sealed by a second seal.

The flexible tube can have a low-friction membrane. The low-friction membrane can have expanded polytetrafluoroethylene. The low friction membrane can be made of polyethylene terathalate or polyethylene. The lube may have one of any different shapes, although a substantially hourglass shape is preferred. The tube can be reinforced using a plurality of filamentary members extending lengthwise along the tube. In order to bias the flexible tube to provide a constricted region an elastic member can be disposed between the flexible tube and the rigid duct or cannula. The tube may also have a plurality of corrugations therein to increase the flexibility of the tube. The flexible tube can be secured to the distal end of the elastic biasing member, and the biasing member and flexible tube are disposed inside the cannula. The proximal end of the biasing member and flexible tube can be secured adjacent the proximal end of the cannula, and may be secured to a housing adjacent the proximal end of the cannula, sometimes known as a cannula housing.

The access port can include a rigid duct or cannula having a distal end positionable within the body cavity. The duct can be extendable through living tissue of the body and having a proximal end positionable outside of the cavity. A flexible tube can be positioned substantially coaxially within the duct and attached thereto. The flexible tube can be secured adjacent the distal and proximal ends of the rigid duct. The tube can have an inner low-friction surface and can define a pocket between the tube and the duct, the pocket being positioned between the opposite ends of the tube. A biasing member can be disposed within the pocket to form a constricted region of the flexible tube between the ends of the tube for contacting an instrument inserted through the duct. The tube can be elastically deformable radially outwardly against the pressure of the biasing member to permit the surgical tool to pass through the duct and into the body cavity. The biasing member can force the constricted region of the membrane to close around the tool to substantially continuously seal the rigid duct while the tool extends therethrough. The flexible tube may but need not seal the rigid duct in the absence of a tool extending through the duct. If the flexible tube does not seal the duct in the absence of a tool, a seal in the absence of a tool can be effected by a separate second seal. The flexible tube can be shaped so as to occupy the central portion of the duct. Pressure relief may be provided to allow gas or fluid between the tube and the duct to vent or escape when a surgical tool is inserted through the duct. Venting may be provided, for example, in the form of slits through the distal portion of the flexible tube or through openings in the duct side wall.

The flexible tube may be a laminate formed with a low-friction membrane forming the inner surface.

### BRIEF DESCRIPTION OF DRAWINGS

The figures form a part of this disclosure in which:

FIG. 1 is an isometric sectional view of a variation of the seal disposed within a cannula;

FIG. 2 is an elevation sectional view of the seal of FIG. 1;

FIG. 3 is an elevation view of a biasing member in accordance with the seal of FIG. 1;

FIG. 4a is an elevation sectional view of a biasing member in accordance with the seal of FIG. 1 disposed in a cannula without a low friction seal membrane:

FIGS. 4b and 4c are various perspective views of the member of FIG. 4a;

FIG. 5 is a partial elevation sectional view in detail showing the distal end of a variation of the low friction membrane secured to the distal end of the biasing member;

FIG. 6a is an elevation sectional view in detail showing the biasing member and low friction membrane secured to the proximal end of the cannula in accordance with the seal of FIG. 1;

FIG. 6b is a close-up view of the top of FIG. 6a;

FIG. 7 is an elevation sectional view of a variation of the seal; and

FIG. 8 is an elevation view of a biasing member in accordance with the seal of FIG. 7.

### DETAILED DESCRIPTION

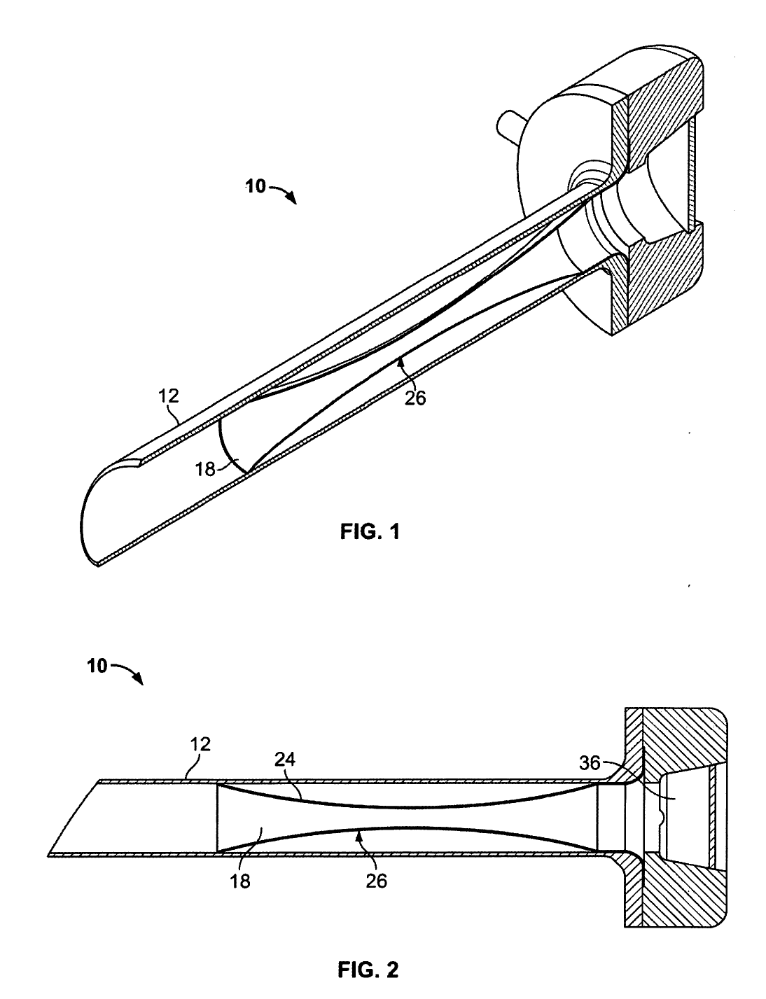

FIG. 1 shows an isometric sectional view of a self sealing surgical access port 10. Access port 10 can comprise a substantially rigid duct or cannula 12 that can be inserted through living tissue into a body cavity wherein the surgical procedure is to be performed in a known manner with the cavity pressurized with a gas such as air, carbon dioxide or nitrous oxide to expand the cavity and provide room therein to insert and manipulate surgical tools during the procedure. Pressurization mat be effected by another access port, not shown, or by providing an insufflation port on the access port 10, also not shown, or by directly insufflating through the access port 10. Tissue can seal against the outer surface of duct 12 by inserting the duct through an opening having a smaller inner diameter than the outer diameter of the duct, thus using the inherent flexibility, resilience and elasticity of the tissue to maintain a seal against the internal pressure with the cavity. The duct can be forced through closed tissue or inserted through an opening already formed in the tissue.

Duct 12 may be cylindrical in shape as shown and may be constructed of nylon as well as other biocompatible materials such as PET, PP, PTFE and polypropylene. Non-cylindrical shapes also are contemplated. A flexible tube 18 can be positioned substantially coaxially within the duct. As shown in cross section in FIG. 2, flexible tube 18 can have an inner surface that can have a low coefficient of friction to permit surgical tools to slide easily over the surface and reduce the propensity of the tools to snag when they are inserted or removed through the duct 12. The low-friction inner surface can be provided by a tubular inner membrane 22 formed from a low-friction material such as expanded polytetrafluoroethylene, polyethylene terathalate or polyethylene. The low-friction material can have low elongation in order to minimize stretching during insertion of an instrument, thereby reducing friction at the instrument interface. The low friction membrane can have high tenacity and/or toughness so that the seal can have high resistance to tearing or puncture. Tubular membranes constructed from the foregoing materials having a thickness of approximately 0.05 mm (0.002 in.) to 0.13 mm (0.005 in.) can exhibit a combination of these characteristics. An elastic biasing member 24 can surround the low-friction inner membrane 22 and can bias the flexible tube to form a constricted region 26. Because the outer membrane is elastic, the flexible tube 18 can elastically deform radially outwardly, for example, to permit surgical tools to pass through the duct and into the cavity. The biasing of the outer membrane can force the inner surface of the low friction flexible tube against the tool 30 and thus continue to provide a fluid seal of the duct 12 even when the tool is inserted through the duct, manipulated within the cavity and withdrawn from the duct.

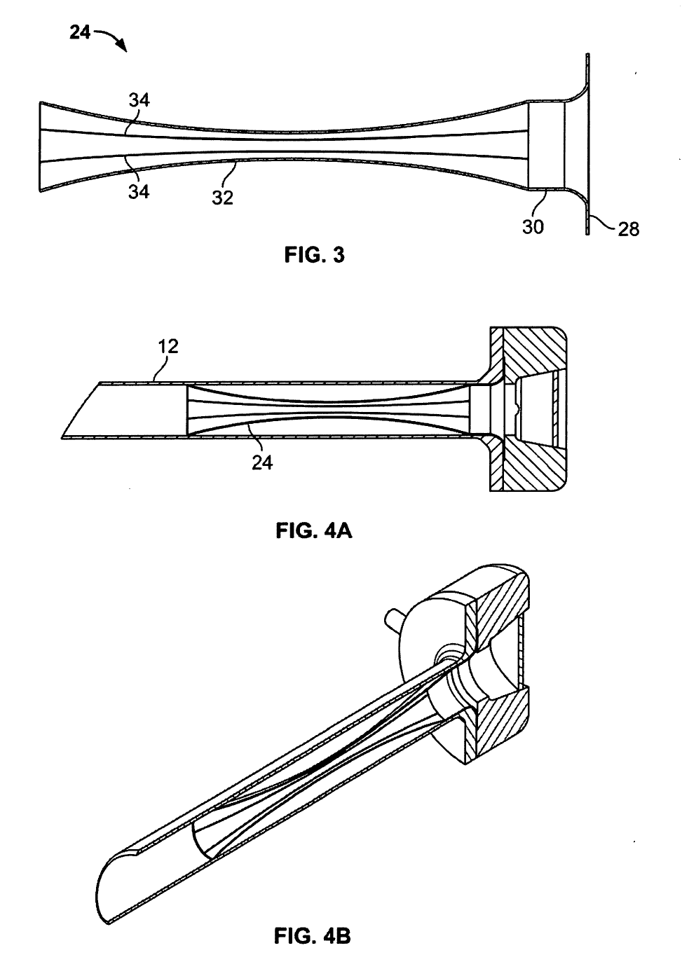

Biasing member 24 is shown side view in FIG. 3. The biasing member 24 can have a proximal flange 28, a proximal cylindrical section 30, and a longitudinally extending biasing region 32. The biasing region 32 can be formed so that in a rest or unstressed position a portion of the biasing region can be narrowed to provide a smaller internal diameter than the diameter of the proximal or distal ends of the biasing member. Longitudinal slits or openings 34 are provided so that portions of the side wall of the biasing member may independently flex radially outward in response to a tool or other object inserted through the biasing member and having a diameter greater than the diameter of the biasing region 32 of the biasing member. The biasing member may be formed of metals such as spring steel or nitinol, molded plastic such as polyester, nylon, PEEK or any other suitable elastomeric material.

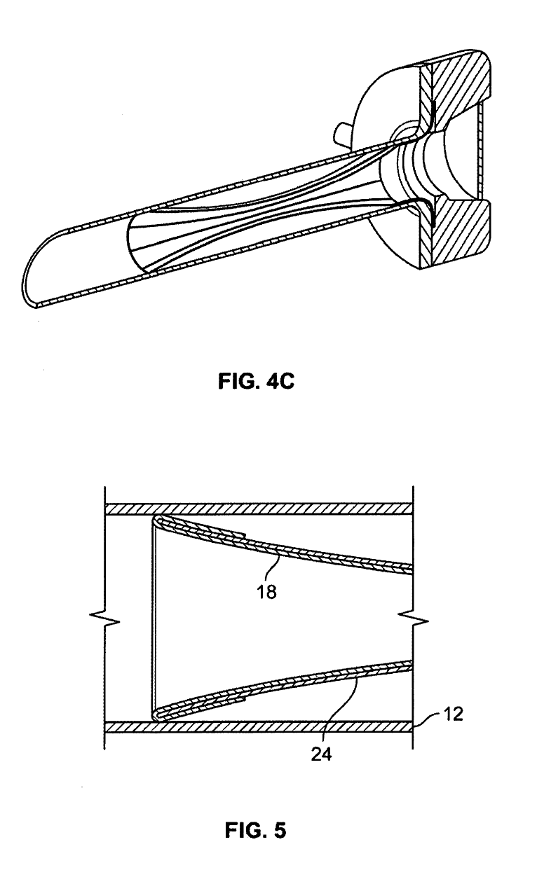

FIGS. 4a through 4c illustrates biasing member 24 disposed within cannula 12 without the low friction seal member. The biasing member can have the relative positioning of the biasing member and cannula rigid duct, as shown. Proximal flange 28 can be nested against the proximal end of the cannula in the cannula housing to constrain the biasing member against forward or backward motion relative to the cannula.

Referring to FIG. 5, the distal end of biasing member 24 is shown inside cannula or duct 12 with low friction seal membrane 18 positioned inside the biasing member. As shown, low friction membrane 18 is disposed against the inner surface of the biasing member, with the distal end of the low friction member extending over and around the end of the biasing member, and secured thereto. In FIG. 5, the low friction member is shown wrapped over and around the distal end of the biasing member. The low friction may be mechanically or otherwise secured relative to the biasing member. For example, the biasing member may have a friction surface, teeth or the like on the inner or outer distal surface thereof to grip and hold the low friction membrane. The low friction membrane may be glued or welded to the distal end of the biasing member.

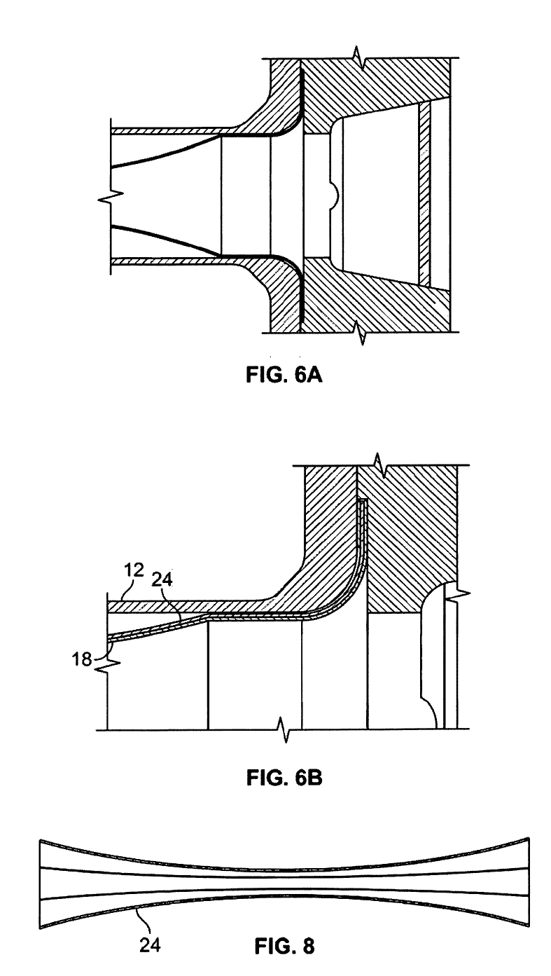

As shown in FIGS. 6a and 6b, the low friction seal membrane similarly can extend over and around the proximal end of the biasing member to hold the low friction membrane in place. More specifically, the low friction membrane is wrapped round the proximal annual flange 28 of the biasing member and may be secured to the biasing member in the same manner as the distal end, e.g., by gluing, welding, frictional engagement with teeth or other surface characteristics, etc. The low friction membrane can be unattached to the biasing member other than at each end of the seal, for example, permitting the elastic member and low friction seal membrane to move independently during insertion and manipulation of instruments.

As shown in FIGS. 1 and 2, the constricted region of the seal can be configured to not seal the duct or cannula when no instrument is inserted through the duct or cannula. Instead or in addition, a zero seal such as a duckbill valve 36 may be provided to seal the duct or cannula when no instrument is inserted through the cannula or duct. An insufflation port (not shown) can be provided as part of the cannula assembly.

In use, the rigid duct or cannula can be inserted through tissue in a known manner until the distal end of the duct or cannula can be disposed inside a body cavity, such as the abdominal. With the cavity inflated and without any instrument inserted through the duct, a seal can be maintained by duckbill valve 36. When a surgical instrument is inserted, low friction membrane 18 can contact the instrument and form a seal therewith. Instruments of varying size, preferably from about 5 mm (0.2 in.) to about 15 mm (0.6 in.) in diameter, may be accommodated by the seal. Thus, the constricted region formed by biasing member 24 biasing the low friction seal radially inward can be configured and dimensioned so that a seal will be formed around the smallest diameter instrument expected to be inserted therethrough, e.g., approximately 5 mm (0.2 in.). When an instrument of larger diameter is inserted, the sections of biasing member 24 can flex radially outward to accommodate the instrument diameter. At the same time, the biasing member sections can maintain exact contact between the flow friction seal membrane and the outer surface of the instrument, thereby maintaining a seal between the low friction membrane and the instrument. The biasing; member and seal membrane can elongate as they expand radially outward, and the biasing member and seal membrane may elongate in the distal direction as an instrument is inserted. The biasing member and seal membrane may move independent of one another along their respective lengths, other than where they are secured together at each end of the seal.

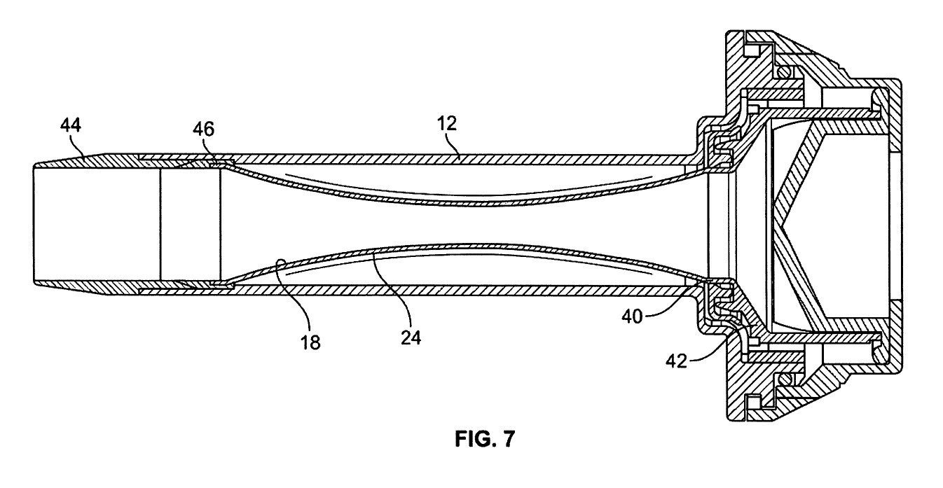

As illustrated in FIGS. 7-8, the low friction seal member 18 may be secured at the distal end to the cannula or a tip associated with the cannula, and at the proximal end thereof to the cannula housing or between the cannula housing and cannula, with the biasing member disposed between the inner surface of the cannula and the outer surface of the low friction membrane.

Referring to FIG. 7, low friction seal membrane 18 can be secured at proximal end 40, for example, by being entrapped between the proximal end of cannula 12 and the inner cannula housing 42. The distal end of the cannula can include a cannula tip insert 44 which can engage the distal end of the cannula 12. The distal end 46 of the low friction seal membrane 18 can be secured between a portion of cannula 12 and distal tip 44. The low friction membrane may be secured to cannula 12 at the proximal and distal ends in a variety of ways, including glue, welding (e.g., ultrasonic welding), friction, mechanical entrapment, or any combination of the foregoing.

Referring to FIG. 8, biasing member 24 can be formed in a rest position to have open ends and a narrowed central region. The biasing member may be made of metal, plastic or other elastomeric materials, for example, sufficient to constrict the central region of the low friction seal member, and having slits or openings so that the biasing member may flex in response to an instrument inserted through the seal. The biasing member and seal membrane can be unattached over their entire length and more completely independently of one another.

Thus, as an instrument is inserted through the seal, the biasing member can maintain the low friction seal member in contact with the instrument, and radially expand in order to permit the instrument to pass through the cannula. The biasing member can be unsecured to the cannula or housing, but rather can be contained in the space between the low friction seal member and the cannula.

Other features also are contemplated, including lubricious coatings on the low friction seal member, alternate zero seals other than a duckbill valve, and the like.

It is apparent to one skilled in the art that various changes and modifications can be made to this disclosure, and equivalents employed, without departing from the spirit and scope of the invention. Elements shown with any embodiment are exemplary for the specific embodiment and can be used in combination with or otherwise on other embodiments within this disclosure.

We claim:

1. A self-sealing surgical port device for providing access to a body cavity for a surgical instrument comprising:
a rigid duct that can have a distal end positionable within the body cavity;
a flexible tube positioned substantially coaxially within the duct, wherein the tube is attached to the duct, and wherein the tube comprises a first seal comprising a membrane exposed to the radially inner surface of the tube, and wherein the tube is elastic:
wherein the tube has open terminal ends and a narrowed central region;
wherein the central region of the tube can be elastically deformable radially outwardly to permit the surgical instrument to pass through the duct and into the body cavity; and
wherein the membrane is configured to close around the instrument to substantially continuously seal the tube while the instrument extends therethrough.

2. The device of claim 1, wherein the tube comprises a low friction surface on the radially inner surface of the tube.

3. The device of claim 2, wherein the membrane has a low-friction surface exposed to the radially inner surface of the tube.

4. The device of claim 1, wherein the duct comprises a second seal.

5. The device of claim 1, wherein the membrane comprises a metal.

6. The device of claim 1, wherein the membrane comprises a plastic.

7. The device of claim 1, wherein the membrane comprises an elastomeric material.

8. The device of claim 1, wherein the membrane has a longitudinal slit configured to flex the membrane in response to inserting of the instrument through the narrow central region.

9. The device of claim 1, wherein the biasing member and seal membrane can be unattached over their entire length and more completely independently of one another.

10. A method of accessing a surgical site comprising:
inserting a seal through tissue and into a body cavity, wherein the radially expanding the seal, wherein a radially expanded configuration of the seal has a longitudinal channel, and wherein the longitudinal channel is configured to allow the passage of a surgical instrument;
inserting the surgical instrument through the longitudinal channel;
affecting tissue in or around the body cavity with the surgical instrument after the surgical instrument is inserted in the longitudinal channel.
